(19) United States
(12) Patent Application Publication
(10) Pub. No.: US 2008/0051676 A1
(43) Pub. Date: Feb. 28, 2008

(54) Extendable Wire Guide system

(76) Inventor: Jeffry S. Melsheimer, Springville, IN (US)

Correspondence Address: BRINKS HOFER GILSON & LIONE/CHICAGO/COOK, PO BOX 10395, CHICAGO, IL, 60610, US

(21) Appl. No.: 11/843,436

(22) Filed: Aug. 22, 2007

Related U.S. Application Data
(60) Provisional application No. 60/840,225, filed on Aug. 24, 2006.

(51) Int. Cl. A61M 25/01

(52) U.S. Cl. 600/585

(57) ABSTRACT

A wire guide includes at least one body portion and a threaded hermaphroditic connector at an end of the at least one body portion adapted to threadably engage a second substantially identical threaded hermaphroditic connector.

### RELATED APPLICATIONS

This application claims the benefit of the filing date under 35 U.S.C. § 119(e) of Provisional U.S. Patent Application Ser. No. 60/840,225, filed Aug. 24, 2006, which is hereby incorporated by reference.

### TECHNICAL FIELD

The present invention relates to a system for connecting a wire guide and an extension wire, and in particular to a threaded hermaphroditic connector of a wire guide that can threadably engage with another threaded hermaphroditic connector of an extension wire.

### BACKGROUND

Wire guides are commonly used in vascular procedures, such as angioplasty procedures, diagnostic and interventional procedures, percutaneous access procedures, or radiological and neuroradiological procedures in general, to introduce a wide variety of medical devices into the vascular system.

As described in U.S. Pat. No. 6,348,041, a wire guide traditionally comprises an elongated core element with one or more tapered sections near the distal end thereof and a flexible helical coil disposed about the distal portion of the core element. The distal extremity of the core element extends through the flexible coil and is secured to a distal end member of the wire guide. Torquing means are provided on the proximal end of the core element to rotate and steer the wire guide while it is being advanced through a patient's vascular system.

In connection with the advancement of the wire guide or once the wire guide has been positioned at a target site inside a patient's body, a wide variety of medical devices may be directed to the target site along the wire guide by simply sliding the device over the wire guide and advancing the device to the distal end of the wire guide. A typical medical device is a catheter, and often a catheter and the wire guide are introduced in a common procedure where the wire guide is advanced a distance in front of the catheter, then the catheter is advanced over the wire guide, followed by a further advancement of the wire guide.

In some cases, it becomes necessary to exchange one medical device for another medical device after the wire guide and the first device have been advanced to the target site. For example, in an angioplasty procedure, it is sometimes necessary to exchange one balloon catheter for another balloon catheter of a different size or shape. In such cases, it is desirable to have a wire guide that is sufficiently long to allow the catheter to be removed over the wire guide and to assist with the placement of another catheter. According to a conventional “over-the-wire” procedure for catheter exchange, the original wire guide is first replaced with a longer exchange wire guide. The original wire guide is removed while the catheter is held in place, and the exchange wire guide is then introduced through the catheter. The exchange wire guide is long enough to extend outside the patient's body for a distance greater than the length of the catheter. The in-place catheter is then removed over the exchange wire guide and a new catheter is inserted. Afterwards, the exchange wire guide is removed and the original wire guide is reinserted. However, such a procedure for catheter exchange is complicated and cumbersome.

In another known procedure for catheter exchange that does not use an exchange wire guide, an extension wire is attached to the proximal end of the original wire guide that extends outside the patient's body. The extension wire extends the overall length of the original wire guide and allows the catheter to be withdrawn and replaced without removing the wire guide. It is also known to provide “rapid-exchange” catheters that are configured for use with shorter wire guides than “over-the-wire” catheters and can be exchanged without using an exchange wire guide or adding an extension wire. In some cases, however, a “rapid-exchange” catheter may not readily available or desirable and an “over-the-wire” catheter has to be used instead. When exchanging a “rapid-exchange” catheter for an “over-the-wire” catheter, the original wire guide may no longer be of sufficient length and an extension wire is desirable.

While several methods for attaching an extension wire to a wire guide are known, for example, using a crimped connector or a pair of mating male and female connectors, there remains a need for an improved system for connecting a wire guide and an extension wire.

### BRIEF SUMMARY

In one aspect of the present invention, a wire guide is provided including at least one body portion and a threaded hermaphroditic connector at an end of the at least one body portion adapted to threadably engage a second substantially identical threaded hermaphroditic connector.

In another aspect of the present invention, a threaded hermaphroditic connector for a wire guide is provided including a tapered shank and at least one helical thread disposed partially about the tapered shank and extending axially outward past an end of the shank. The helical thread includes an axial opening adapted to receive a tapered shank of a second substantially identical threaded hermaphroditic connector.

### BRIEF DESCRIPTION OF THE DRAWINGS

FIG. 1 shows an illustrative wire guide and extension wire;

FIG. 2 shows a fragmented sectional view of the wire guide of FIG. 1 with a single-thread hermaphroditic connector according to the invention;

FIG. 3 shows a side elevational view of the threaded hermaphroditic connector of FIG. 2;

FIG. 4 shows a partial perspective view of the wire guide of FIG. 2 and the extension wire of FIG. 1 with a second substantially identical threaded hermaphroditic connector according to the invention;

FIG. 5 shows a partial perspective view of the wire guide and extension wire of FIG. 4 with the threaded hermaphroditic connector of the wire guide partially engaged with the second substantially identical threaded hermaphroditic connector of the extension wire;

FIG. 6 shows a side elevational view of a double-thread hermaphroditic connector according to the invention; and

FIG. 7 shows a fragmented sectional view of a two-piece modular wire guide having a pair of hermaphroditic connectors according to the invention.

### DETAILED DESCRIPTION

In accordance with an embodiment of the present invention, an extendable wire guide system includes a wire guide having a threaded hermaphroditic connector at an end thereof and an extension wire having another substantially identical threaded hermaphroditic connector at an end thereof for selectively connecting the extension wire to the wire guide. The term “hermaphroditic” as used herein and throughout to describe embodiments of the invention is intended to refer to a design for a connector that is neither male nor female and can mate with another connector of similar design. Also, the term “proximal” refers to a portion of the wire guide closest to a physician when placing a wire guide in a patient, and the term “distal” refers to a portion of the wire guide closest to the end inserted into the patient's body.

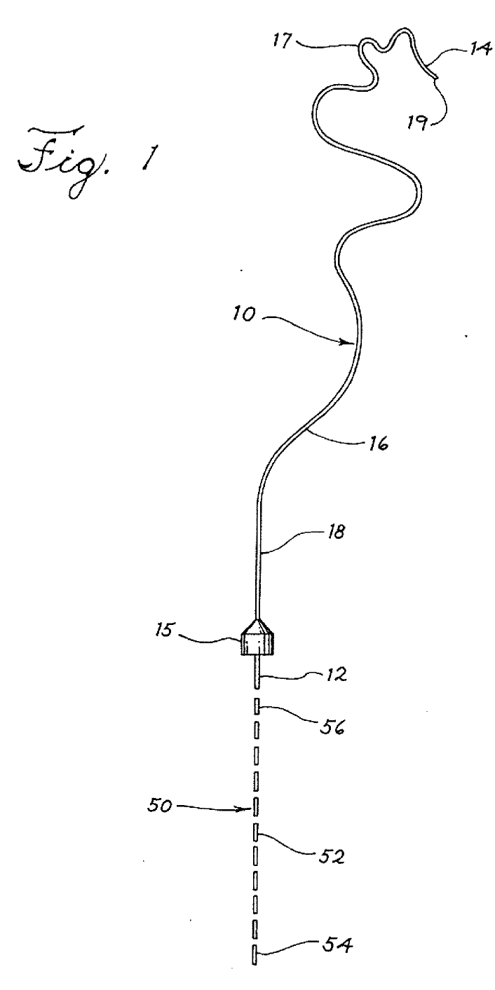

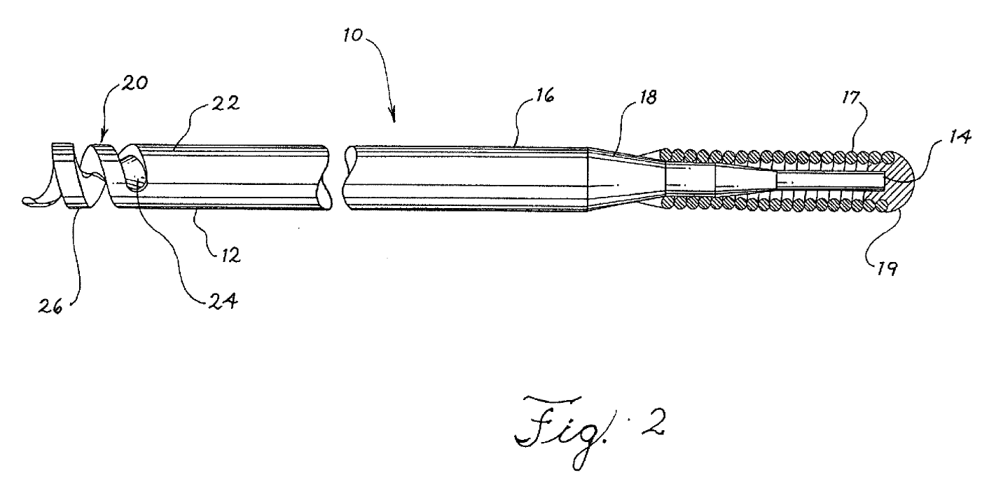

Referring now more specifically to the drawings, an illustrative wire guide that can incorporate a threaded hermaphroditic connector according to the present invention is shown at 10 in FIGS. 1 and 2. The wire guide 10 includes a proximal end 12 that is kept outside the patient's body and a flexible distal end 14 capable of being advanced to a target site in the vascular system. A body portion 16 extends from the proximal end 12 towards the distal end 14 and carries near the proximal end a handle 15 releasably secured to the wire guide. The body portion 16 can have, for example, a circular cross-sectional area that diminishes gradually or stepwise at increasing distance from the proximal end 12 of the wire guide 10. The wire guide 10 typically has a length in the range of 50-300 cm and a maximum outer diameter in the range of 0.204-1.072 mm (0.008-0.042 inches), although those skilled in the art will readily recognize that the wire guide 10 can have other shapes and dimensions.

The body portion 16 may include an elongated solid shaft 18 and a coiled distal portion 17. The solid shaft 18 is of a suitable metallic material such as medical grade stainless steel or Nitinol. It will be appreciated that conventional drawing techniques can be used to manufacture the solid shaft 18, for example, as a core wire of constant diameter. The core wire can be centerless ground to provide the solid shaft 18 with a decreasing cross-sectional area at increasing distance from the proximal end 12. Alternatively, the core wire for the solid shaft 18 can be drawn in stages to provide a cross-sectional area that diminishes stepwise at increasing distance from the proximal end 12.

The coiled distal portion 17 is fixed at its proximal end onto the distal end of the solid shaft 18. The coiled distal portion 17 can be made, for example, of a linear elastic material, such as stainless steel, titanium or tantalum, or a superelastic alloy, such as Nitinol, and can be fixed to the solid shaft 18 in a suitable manner as is known in the art, for example, by welding, soldering, or a brazed joint. Alternatively, it will be appreciated that the coiled portion 17 can continue from the distal end 14 to the proximal end 12 of the wire guide 10, and the use of a solid shaft is made superfluous. The coiled distal portion 17 ends distally at a distal end member 19, which is a member having an atraumatic front end termination, such as a rounded front or a front of very flexible material or very flexible configuration. For example, end member 19 can be a solder ball, or a sphere that is laser welded onto the distal end of the coiled portion 17, and can include a soft coil of radiopaque material (not shown).

Also shown in FIG. 1 is an illustrative extension wire 50 that can incorporate a second substantially identical threaded hermaphroditic connector according to the present invention for selectively connecting to the wire guide 10. The extension wire 50 generally includes a shaft member 52, for example a stainless steel shaft, provided with a proximal end 54 and a distal end 56. The shaft member 52 can have a constant diameter or can include several segments of reduced diameter such that the extension wire 50 tapers to a smaller diameter toward its distal end 56. Preferably, the outer diameter of the extension wire 50 at the distal end 56 is substantially the same as the outer diameter of the wire guide 10 at the proximal end 12.

In the embodiment illustrated in FIGS. 2-5, the wire guide 10 has a single-thread hermaphroditic connector 20 at its proximal end 12. The threaded hermaphroditic connector 20 includes a base member 22 disposed adjacent the body portion 16. The base member 22 can have any suitable cross-sectional shape. Preferably, the base member 22 has the same cross-sectional shape as the body portion 16 and its outer diameter is approximately equal to the outer diameter of the body portion 16 at the proximal end 12.

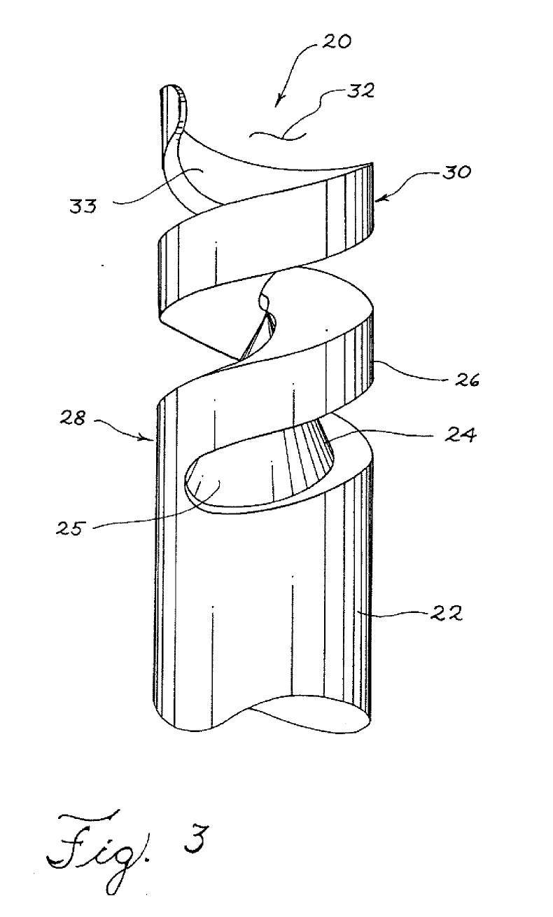

The threaded hermaphroditic connector 20 also includes a tapered shank 24 and a helical thread 26 disposed partially about the shank 24. As shown in greater detail in FIG. 3, the shank 24 and the helical thread 26 extend out from the base member 22. The helical thread 26 extends out from the base member 22 a predetermined distance greater than the height of the tapered shank 24. For example, the axial length of the thread 26 may be about twice the height of the tapered shank 24, which may be approximately equal to or greater than the diameter of the tapered shank 24.

The tapered shank 24 has a positive conical outer surface 25 tapering inward from the base member 22. The helical thread 26 includes a bottom section 28 disposed about the tapered shank 24 and a top section 30 that extends out past the tip of the tapered shank 24. Preferably, the end of the top section 30 extending out past the tip of the tapered shank 24 has a rounded profile so as to be atraumatic. The top section 30 of the helical thread 26 includes a longitudinally extending opening 32 therethrough. The inner walls of the opening 32 form a negative conical interior surface 33 having a shape complementary to the shape defined by the positive conical outer surface of the tapered shank of another substantially identical threaded hermaphroditic connector, such that the opening 32 can matingly receive the tapered shank of the other threaded hermaphroditic connector. It should be understood, however, that the invention is not limited to these conical shapes for the tapered shank 24 and the opening 32. For example, in an alternative embodiment, the outer surface of the tapered shank 24 defines a polyhedral shape, such as a pyramid tapering inward from the base member 22, while the interior surface of the opening 32 defines a complementary shape that can matingly receive the tapered shank of another threaded hermaphroditic connector.

The helical thread 26 can have any suitable outer diameter, thread pitch, pitch angle, thread depth, and thread angle. The thread pitch is the distance parallel to the center axis of the helical thread 26 between corresponding points on adjacent crests of the thread 26. The pitch angle is the included angle between the longitudinal axis of the threaded hermaphroditic connector 20 and the helical thread 26. The thread depth is the distance, measured radially, between the crest and the root of the thread 26. The thread angle is the included angle between the sides of the thread.

In the present embodiment, the helical thread 26 has a constant outer diameter less than or approximately equal to the outer diameter of the wire guide 10 at the proximal end 12. The thread pitch of the helical thread 26 is approximately equal to or greater than the outer diameter of the helical thread 26, the pitch angle is between about 45 degrees and about 72 degrees, and more preferably about 63 degrees, the thread depth is approximately equal to one-half the thread pitch, and the thread angle is approximately zero degrees (i.e., a square thread). However, it will be readily understood that the helical thread 26 can have other dimensions, for example, a tapered outer diameter or a different thread pitch, pitch angle, thread depth, and thread angle.

The threaded hermaphroditic connector 20 can be manufactured using known manufacturing techniques, such as precision machining or investment casting for creating high precision metallic parts with complex geometry. The threaded hermaphroditic connector 20 is formed of a suitable material such as stainless steel or Nitinol and is secured to the body portion 16 at the proximal 12 end of the wire guide in any suitable manner as, for example, by welding or soldering. Alternatively, the threaded hermaphroditic connector 20 can be formed integrally on the body portion 16 in extension of the proximal end 12 of the wire guide. If the body portion 16 includes the solid shaft 18, then the threaded hermaphroditic connector 20 can be formed integrally at the proximal end of the solid shaft 18, for example, using a “Swiss machining” process that is capable of turning and milling small precision parts.

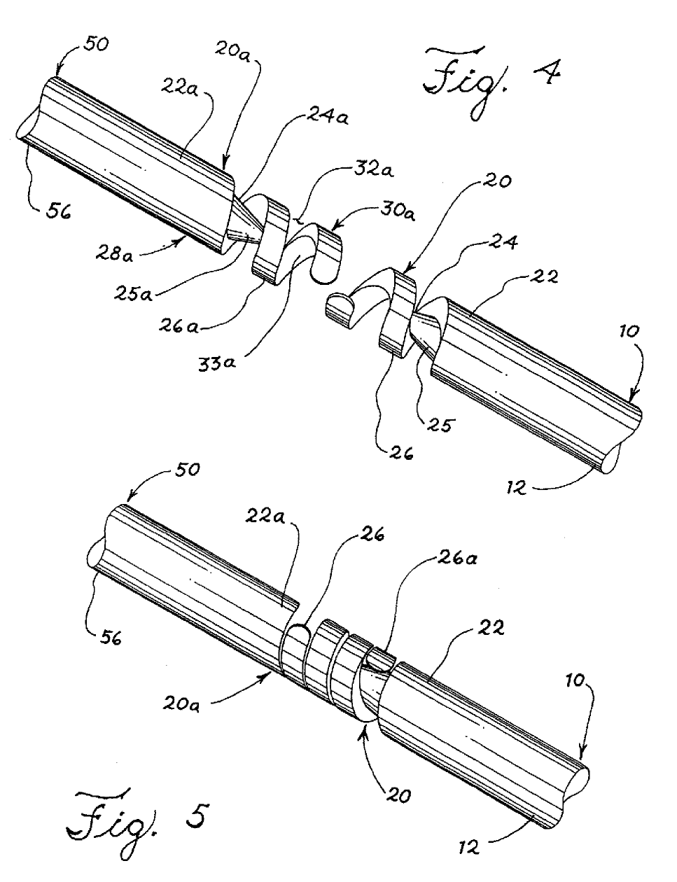

As shown in FIG. 4, a single-thread hermaphroditic connector 20a is also disposed at the distal end 56 of the extension wire 50 for threadably engaging the single-thread hermaphroditic connector 20 at the proximal end 12 of the wire guide 10. The hermaphroditic connector 20a is made of a suitable material such as stainless steel or Nitinol, and is secured to the shaft member 52 at its distal end 56 in any suitable manner as, for example, by welding or soldering. Alternatively, the hermaphroditic connector 20a can be formed integrally on the shaft member 52 in extension of the distal end 56.

The single-thread hermaphroditic connector 20a is substantially identical to and has the same dimensions and features as the single-thread hermaphroditic connector 20, including a tapered shank 24a and a helical thread 26a extending out from a base member 22a. The tapered shank 24a has a positive conical outer surface 25a tapering inward from the base member 22a. The helical thread 26a includes a bottom section 28a disposed about the tapered shank 24a and an atraumatic top section 30a that extends out past the tip of the tapered shank 24a. The top section 30a of the helical thread 26a includes a longitudinally extending opening 32a therethrough. The inner walls of the opening 32a form a negative conical interior surface 33a having a shape complementary to the shape defined by the positive conical outer surface 25 of the tapered shank 24 of the threaded hermaphroditic connector 20, such that the opening 32a can matingly receive the tapered shank 24 of the threaded hermaphroditic connector 20.

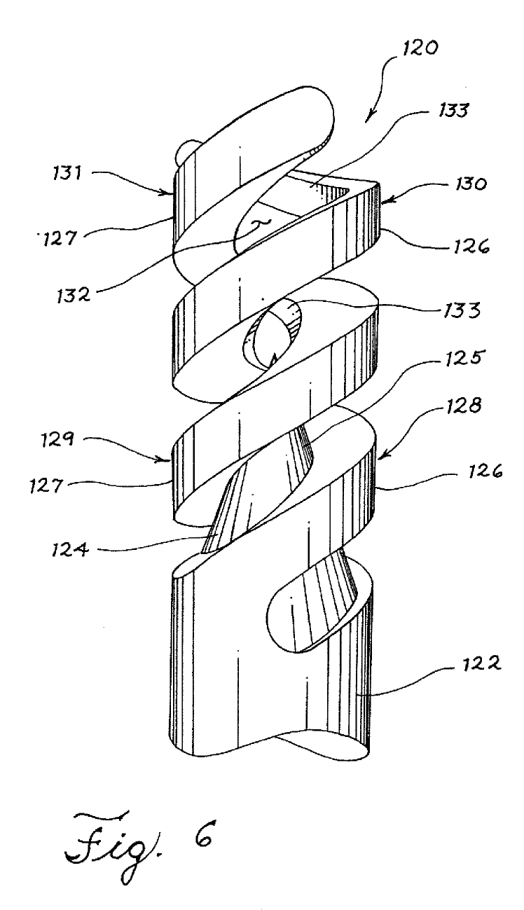

It will be appreciated from the foregoing that the extension wire 50 can be selectively connected to the wire guide 10 by rotating the extension wire 50 relative to the wire guide 10 so as to threadably engage the hermaphroditic connector 20 with the hermaphroditic connector 20a as shown in FIG. 5. The direction of rotation for the extension wire 50 necessary to engage the threaded hermaphroditic connectors 20, 20a will depend on whether the helical threads 26, 26a have a right-handed configuration, i.e., sloping upward to the right, or a left-handed configuration, i.e., sloping upward to the left. Thus, a right-handed configuration for the helical threads 26, 26a requires a clockwise rotation of the extension wire 50, while a left-handed configuration requires a counterclockwise rotation. Initially, the rotation causes the helical threads 26, 26a to engage and travel axially toward each other. Further rotation causes the negative conical interior surface 33 of the threaded hermaphroditic connector 20 to engage the tapered shank 24a of the threaded hermaphroditic connector 20a, and also the negative conical interior surface 33a of the threaded hermaphroditic connector 20a to engage the tapered shank 24 of the threaded hermaphroditic connector 20.

The threaded connection formed by the threaded hermaphroditic connectors 20, 20a is sufficiently strong to allow a user to steer and maneuver the wire guide 10 through a patient's vascular system without the connectors disengaging. In addition, the openings 32, 32a may be sized to provide an interference fit with the tapered shanks 24a, 24 when engaged. For example, the negative conical interior surfaces 33, 33a may have a slightly different taper angle or a slightly smaller major diameter than the positive conical outer surface 25a, 25. This permits the openings 32, 32a to matingly receive the tapered shanks 24a, 24 in an interference relation. Alternatively, the negative conical interior surfaces 33, 33a of the openings 32, 32a and the positive conical outer surface 25a, 25 of the tapered shanks 24a, 24 may have a set of protuberances and/or recesses, such as teeth, undulations or other raised features, formed thereon that frictionally engage each other so as to provide a more secure connection between the threaded hermaphroditic connectors 20, 20a. In yet another embodiment, it will be understood that tapered shanks 24, 24a having polyhedral outer surfaces, as described above, will provide an interference fit with the openings 32, 32a having complementary interior surfaces when engaged.

The threaded hermaphroditic connector 20 of the wire guide 10 and the threaded hermaphroditic connector 20a of the extension wire 50 thread into each other such that the connection formed between them forms a smooth joint between the wire guide 10 and the extension wire 50. Preferably, the outer diameter of the threaded hermaphroditic connectors 20, 20a is less than or approximately equal to the outer diameter of the extension wire 50 at its distal end 56 and the outer diameter of the wire guide 10 at its proximal end 12. In this way, the connection formed by the threaded hermaphroditic connectors 20, 20a will also have an outer diameter smaller than the outer diameter of the extension wire 50 and the wire guide 10, and will not obstruct or interfere with the travel of a catheter over the wire guide 10 and the extension wire 50.

In addition, the extension wire 50 may also be releasably connected to the wire guide 10. In particular, the extension wire 50 and the wire guide 10 can be detached by rotating the extension wire 50 relative to the wire guide 10 in a second direction opposite to the first direction necessary for engagement. From the foregoing, it will be appreciated that the extension wire 50 and the wire guide 10 can be reconnected and detached multiple times.

According to one illustrative use, the wire guide 10 can be percutaneously introduced into the vascular system of a patient with a dilatation catheter through an introducer (not shown). The distal end 14 of the wire guide is advanced beyond the distal tip of the dilatation catheter while the latter is held in place. Then, the wire guide 10 is advanced into the selected artery. The distal end 14 of the wire guide is preferably advanced through the lesion and beyond it, in order to permit the balloon portion of the dilatation catheter to be positioned within the lesion over a more supportive section of the wire guide. Once in position, the wire guide 10 is held in place and the dilatation catheter is advanced until the inflatable balloon thereof is within the lesion. The proximal end 12 of the wire guide 10 is kept outside the patient's body.

To exchange catheters, the wire guide 10 is extended by manually threading the hermaphroditic connector 20a on the distal end 56 of the extension wire 50 and the threaded hermaphroditic connector 20 at the proximal end 12 of the wire guide. When the wire guide 10 and the extension wire 50 are threadably connected together, the dilatation catheter can then be withdrawn from the patient's body over the extended wire guide system while maintaining the position of the wire guide 10.

A new dilatation catheter may then be introduced over the extension wire 50 and advanced along the wire guide 10 within the patient's body until the balloon crosses the lesion. Once the proximal end of the new balloon catheter has advanced beyond the threaded connection between the threaded hermaphroditic connectors 20, 20a, the extension wire 10 can be removed by rotating the threaded hermaphroditic connector 20a and then pulling the extension wire 50 and wire guide 10 apart without disturbing the position of the wire guide 10 in the patient's body.

If desired, the extension wire 50 may have a hermaphroditic connector 20a attached at each end. In this way, either the proximal end 54 or the distal end 56 of the extension wire 50 could be threadably connected to the at its proximal end 12 of the wire guide 10, thereby simplifying the procedure for exchanging catheters and minimizing the risk of interruptions to the procedure caused by an attempt to thread the wrong end of the extension wire 50 to the proximal end 12 of the wire guide 10.

It may also be desirable to provide the wire guide 10 with an atraumatic back end termination member (not shown) that attaches to the proximal end 12 when the extension wire 50 is not being used. For example, the back end termination member can include a short section of very flexible material or very flexible configuration secured to a threaded hermaphroditic connector substantially identical to the hermaphroditic connector 20 for selectively connecting the back end termination to the wire guide 101

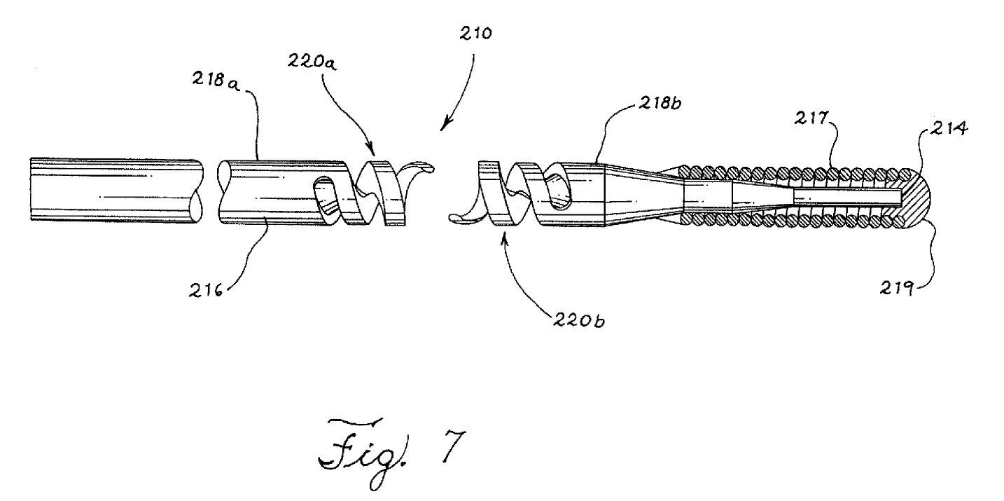

In another embodiment, a double-thread hermaphroditic connector 120 as shown in FIG. 6 is provided for the wire guide 10. The threaded hermaphroditic connector 120 includes a base member 122. The base member 122 can have any suitable cross-sectional shape. Preferably, the base member 122 has the same cross-sectional shape as the body portion 16 and its outer diameter is approximately equal to the outer diameter of the body portion 16 at the proximal end 12.

The threaded hermaphroditic connector 120 also includes a tapered shank 124 and a pair of helical threads 126, 127 disposed partially about the shank. The shank 124 and the helical threads 126, 127 extend out from the base member 122. The helical threads 126, 127 extend out from the base member 122 a predetermined distance greater than the height of the tapered shank 124. For example, the axial length of the threads 126, 127 may be about twice the height of the tapered shank 124, which may be approximately equal to or greater than the diameter of the tapered shank 124. The helical threads 126, 127 extend out from opposite locations on the base member 122 such that they differ by a translation along their common axis to form a double helix configuration.

The tapered shank 124 has a positive conical outer surface 125 tapering inward from the base member 122. The helical threads 126, 127 include bottom sections 128, 129 disposed about the tapered shank 124 and top sections 130, 131 that extend out past the tip of the tapered shank 124. The top sections 130, 131 of the helical threads 126, 127 include a longitudinally extending opening 132 therethrough. The inner walls of the opening 132 form a negative conical interior surface 133 having a shape complementary to the shape defined by the positive conical outer surface of the tapered shank of another substantially identical threaded hermaphroditic connector, such that the opening 132 can matingly receive the tapered shank of the other threaded hermaphroditic connector.

The helical threads 126, 127 have the same outer diameter, thread pitch, pitch angle, thread depth, and thread angle. In the present embodiment, the helical threads 126, 127 preferably have a constant outer diameter less than or approximately equal to the outer diameter of the wire guide 10 at the proximal end 12. The thread pitch of the helical threads 126, 127 is approximately equal to or greater than the outer diameter of the helical threads 126, 127, the pitch angle is between about 45 degrees and about 72 degrees, and more preferably about 63 degrees, the thread depth is approximately equal to one-fourth the thread pitch, and the thread angle is approximately zero degrees (i.e., a square thread). However, it will be readily understood that the helical threads 126, 127 can have other dimensions, for example, a tapered outer diameter or a different thread pitch, pitch angle, thread depth, and thread angle.

The double-thread hermaphroditic connector 120 is disposed at the proximal end 12 of the wire guide 10 for threadably engaging another substantially identical double-thread hermaphroditic connector (not shown) at the distal end 56 of the extension wire 50 so as to attach the extension wire 50 to the wire guide 10. It will be appreciated from the foregoing that the extension wire 50 can be rotated relative to the wire guide 10 in order to threadably engage the double-thread hermaphroditic connector 120 of the wire guide 10 with the other double-thread hermaphroditic connector of the extension wire 50. A right-handed configuration for the helical threads 126 requires a clockwise rotation of the extension wire 50, while a left-handed configuration requires a counterclockwise rotation. Initially, the rotation causes the helical threads 126, 127 of the double-thread hermaphroditic connector 120 to engage the helical threads of the substantially identical double-thread hermaphroditic connector and to travel axially toward each other. Further rotation causes the negative conical interior surface 133 of the double-thread hermaphroditic connector 120 to engage the tapered shank of the substantially identical double-thread hermaphroditic connector, and also the negative conical interior surface of the substantially identical double-thread hermaphroditic connector to engage the tapered shank 124 of the double-thread hermaphroditic connector 120.

The opening 132 of the double-thread hermaphroditic connector 120 may be sized to provide an interference fit with the tapered shank of the substantially identical double-thread hermaphroditic connector when engaged. Alternatively, the negative conical interior surface 133 and the positive conical outer surface 125 can have a set of protuberances, such as teeth, undulations or other raised features, formed thereon that frictionally engage the protuberances of the substantially identical double-thread hermaphroditic connector.

In another embodiment, one type of an extendable wire guide system according to the present invention can be a modular wire guide system, including a wire guide having a pair of threaded hermaphroditic connectors for selectively connecting a desired distal tip thereto. For example, as shown in FIG. 7, the body portion 216 of a wire guide 210 may have a two-piece construction, including a proximal member 218a having a threaded hermaphroditic connector 220a at its distal end and a distal member 218b having a substantially identical threaded hermaphroditic connector 220b at its proximal end. The coiled distal portion 217, which ends distally at a distal end member 219, may be fixed onto the distal member 218b to provide a flexible distal end 214 for the wire guide 210. In this way, a user can selectively connect different distal members 218b, including different coiled distal portions 217, to the proximal member 218a of the wire guide so as to change the distal tip characteristics of the wire guide. For example, the flexibility, torqueability and pushability of the wire guide 210 may vary depending upon the material properties and shape of the different distal portions 217.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. Thus, by way of example and not of limitation, the threaded hermaphroditic connector according to the present invention may include three or more helical threads.

It is therefore intended to include within the invention all such variations and modifications as fall within the scope of the appended claims and equivalents thereof.

What is claimed is:

1. A wire guide comprising:
at least one body portion;
a threaded hermaphroditic connector at an end of the at least one body portion adapted to threadably engage a second substantially identical threaded hermaphroditic connector.

2. The wire guide of claim 1 wherein the threaded hermaphroditic connector includes a tapered shank and at least one helical thread disposed partially about the tapered shank and extending axially outward past an end of the shank.

3. The wire guide of claim 2 wherein the at least one helical thread includes an axial opening having a shape complementary to the shape of a tapered shank of the second substantially identical threaded hermaphroditic connector.

4. The wire guide of claim 3 wherein the at least one helical thread includes a bottom section disposed about the tapered shank and a top portion that extends axially outward past the end of the shank and includes the axial opening.

5. The wire guide of claim 3 wherein the tapered shank has a positive conical outer surface and the axial opening forms a negative conical interior surface.

6. The wire guide of claim 5 wherein at least one of the negative conical interior surface and the positive conical outer surface has a first set of protuberances and recesses formed thereon that are adapted to frictionally engage a second set of protuberances and recesses of the second threaded hermaphroditic connector.

7. The wire guide of claim 3 wherein the tapered shank has a polyhedral outer surface.

8. The wire guide of claim 3 wherein the axial opening is sized smaller than the tapered shank and is adapted to provide an interference fit with the tapered shank of the second threaded hermaphroditic connector when engaged.

9. The wire guide of claim 3 wherein the axial opening has a different taper angle than the tapered shank and is adapted to provide an interference fit with the tapered shank of the second threaded hermaphroditic connector when engaged.

10. The wire guide of claim 2 wherein the at least one helical thread includes a pair of helical threads arranged in a double helix configuration.

11. The wire guide of claim 1 wherein the threaded hermaphroditic connector and the body portion are a unitary construction.

12. The wire guide of claim 1 wherein the end of the at least one body portion corresponds to a proximal end of the wire guide.

13. The wire guide of claim 1 further comprising two body portions, wherein the threaded hermaphroditic connector is secured at a distal end of a first body portion and is adapted to threadably engage a second substantially identical threaded hermaphroditic connector secured to a proximal end of a second body portion.

14. A threaded hermaphroditic connector for a wire guide comprising:
a tapered shank and at least one helical thread disposed partially about the tapered shank and extending axially outward past an end of the shank,
wherein the at least one helical thread includes an axial opening adapted to receive a tapered shank of a second substantially identical threaded hermaphroditic connector.

15. The connector of claim 14 wherein the helical thread includes a bottom section disposed about the tapered shank and a top portion that extends axially outward past the end of the shank and includes the axial opening.

16. The connector of claim 14 wherein the tapered shank has a positive conical outer surface and the axial opening has a negative conical interior surface.

17. The wire guide of claim 16 wherein at least one of the negative conical interior surface and the positive conical outer surface has a set of protuberance formed thereon.

18. The wire guide of claim 14 wherein the tapered shank has a polyhedral outer surface.

19. The wire guide of claim 14 wherein the axial opening is sized smaller than the tapered shank.

20. The wire guide of claim 14 wherein the axial opening has a different taper angle than the tapered shank.

21. The connector of claim 14 wherein the at least one helical thread comprises a pair of helical threads arranged in a double helix configuration.
